(19) United States
(12) Patent Application Publication
(10) Pub. No.: US 2014/0378963 A1
(43) Pub. Date: Dec. 25, 2014

(54) TREATMENT OF UTERINE FIBROIDS BY ARTERIAL ABLATION

(71) Applicant: GYRUS ACMI, INC., D.B.A. OLYMPUS SURGICAL TECHNOLOGIES AMERICA, Southborough, MA (US)

(72) Inventors: Kester BATCHELOR, Mound, MN (US); Tracey DOBBS, Delano, MN (US); Jyue Boon LIM, New Brighton, MN (US); Nikhil MURDESHWAR, Maple Grove, MN (US); Tsuyoshi HAYASHIDA, Maple Grove, MN (US)

(73) Assignee: GYRUS ACMI, INC., D.B.A. OLYMPUS SURGICAL TECHNOLOGIES AMERICA, Southborough, MA (US)

(21) Appl. No.: 13/922,485

(22) Filed: Jun. 20, 2013

(51) Int. Cl. A61B 18/14

(52) U.S. Cl. CPC A61B 18/1477; A61B 2018/1425
USPC 606/41

(57) ABSTRACT

Methods and apparatus for ablating blood vessels in treatment of uterine fibroids. A monopolar electrode provided on a needle probe is positioned in an artery supplying blood to a uterine fibroid. Energy is supplied to the electrode to ablate the artery. In this way, the uterine fibroid and the surrounding tissues remain relatively undamaged, and the uterine fibroid, deprived of its blood source, is allowed to necrose and eventually shrink.

### BACKGROUND

This disclosure relates to methods and apparatus for ablating arteries feeding into uterine fibroids.

Uterine fibroids are the most common pelvic tumor in women, affecting approximately one quarter of women during their reproductive years. Uterine fibroids are generally noncancerous, but may potentially lead to infertility or cause adverse effects if they occur during pregnancy. Typical symptoms include abnormal bleeding, pressure, or pain.

Uterine fibroids are categorized based on location on the uterus. Sub-mucosal fibroids form on the inside wall of the uterus; sub-serosal fibroids form on the outside wall of the uterus; intra-mural fibroids form within the wall of the uterus; and pedunculated fibroids are connected to the inside or outside wall of the uterus.

Currently uterine fibroid treatments include both pharmaceutical and surgical techniques. Pharmaceutical treatments often do not adequately treat the symptoms of uterine fibroids, ultimately necessitating surgical intervention. Surgical techniques include hysterectomy, myomectomy, endometrial ablation, myolysis, and uterine artery occlusion. In addition, interventional radiology and high frequency focused ultrasound techniques exist for the treatment of uterine fibroids.

All of these treatment techniques suffer from shortcomings, such as the risk of relapse, infertility, and applicability to only one or a few types of uterine fibroids.

### SUMMARY

Some uterine fibroid treatments make use of electrodes emitting radio frequency energy to ablate the vessels supplying blood to uterine fibroids. For example, some devices place an electrode near the blood vessels, and emit radio frequency energy toward the blood vessels. This presents the problem that too much surrounding tissue can be ablated.

One might consider a uterine fibroid treatment that makes use of a bipolar electrode placed within the blood vessel supplying blood to the uterine fibroid. However, because bipolar devices typically use lower voltages, their affect may be too localized to effectively ablate the blood vessel.

In order to address these difficulties, aspects of the invention relate to methods and apparatus for ablating blood vessels supplying uterine fibroids with blood. A monopolar electrode is placed within a blood vessel leading to the uterine fibroid. A return electrode may be placed elsewhere, for example, on the patient's abdomen. By supplying radio frequency energy from the monopolar electrode placed in the blood vessel, the blood vessel can be ablated without also ablating the uterine fibroid or other surrounding tissue. The fibroid, starved of blood supply and its associated nutrients, will then necrose and shrink over time.

In the case that a uterine fibroid is supplied by more than one blood vessel, the procedure is performed for each blood vessel.

According to some embodiments, the procedure is performed using a needle probe having the monopolar electrode at and/or adjacent to the sharp distal end of the needle probe. The electrode is positioned within the blood vessel by piercing the blood vessel with the sharp distal end of the needle probe, and then inserting at least the distal end of the probe into the vessel until the monopolar electrode is located at the desired position within the blood vessel. Energy then is supplied to the monopolar electrode to ablate the blood vessel. According to some embodiments, at least the distal end of the needle probe has a diameter of about 1-2 mm.

According to some embodiments of the invention, the blood vessel is occluded prior to ablation so that the flow of blood through the blood vessel is stopped prior to the ablation. For example, external pressure can be applied to the vessel to occlude the vessel. Alternatively, the vessel can be occluded after the vessel has been pierced by the needle probe. This may be done by inflating a balloon in the blood vessel, which may be integral with the needle probe. For example, after piercing the blood vessel with the distal end of the needle probe, a balloon can be deployed through the distal end into the blood vessel. Once inflated within the vessel, the vessel will be occluded, and then vessel ablation can start.

### BRIEF DESCRIPTION OF THE DRAWINGS

Exemplary embodiments will be described in detail with reference to the following drawings in which:

FIG. 1 illustrates various locations of uterine fibroids;

FIG. 2 illustrates an enlarged view of a fibroid having one of its blood vessels ablated by a needle probe according to an embodiment of the invention; and

FIG. 3 illustrates an enlarged view of a needle probe which can be used in an ablative procedure.

### DETAILED DESCRIPTION OF EMBODIMENTS

The following exemplary embodiments are described below with reference to the figures in the context of uterine fibroid treatment, and in particular arterial ablation of uterine fibroids,

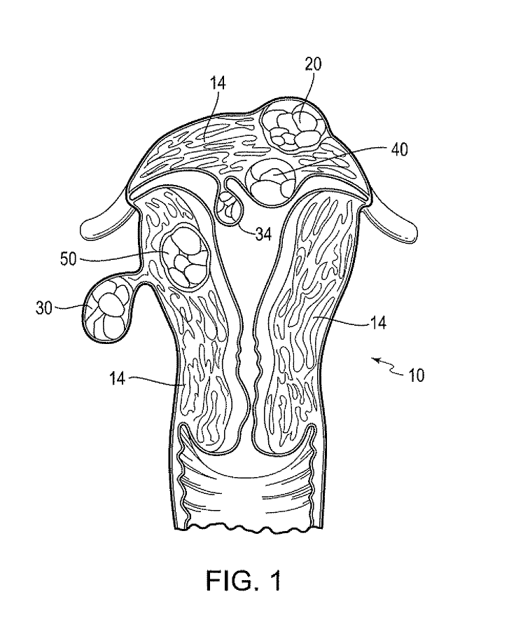

FIG. 1 illustrates different anatomical locations of uterine fibroids that can potentially afflict a patient. A sub-mucosal fibroid 40 is located on the inside wall of the uterus 10. A sub-serosal fibroid 20 is located on the outside wall of the uterus 10. An intra-mural fibroid 50 is located within the wall 14 of the uterus 10. A pedunculated fibroid 30 is attached to the outer wall of the uterus 10. Because it is attached to the outer wall of the uterus 10, fibroid 30 more specifically is known as a pedunculated sub-serosal fibroid. Fibroid 34 is known as a pedunculated sub-mucosal fibroid because it is attached to the inner wall of the uterus 10.

The location of a patient's fibroid(s) is first determined by one or more known imaging techniques. For example, ultrasonic imaging (known as “ultrasound”) can be performed using a transducer placed externally of the patient's body or located within the uterus, for example, at the end of a transcervically inserted ultrasonic probe. The feature of Doppler employed with ultrasound allows for the tracking of blood flow, and thus is very suitable. MRI also could be used.

Once the location of the (or each) fibroid has been determined, the surgeon will determine how to access the fibroid(s) to perform arterial ablation of the blood vessels supplying blood to the fibroid(s). For example, sub-mucosal fibroids and pedunculated sub-mucosal fibroids typically are accessed transcervically, whereas sub-serosal fibroids, pedunculated sub-serosal fibroids and intra-mural fibroids typically are accessed from the pelvic cavity (i.e., laproscopically accessed). However, the manner of accessing each fibroid also depends on the desired outcome of the surgery (e.g., fertility, resolution of the patient's symptoms, etc.), the size of each fibroid, as well as the location of other fibroids within the uterus.

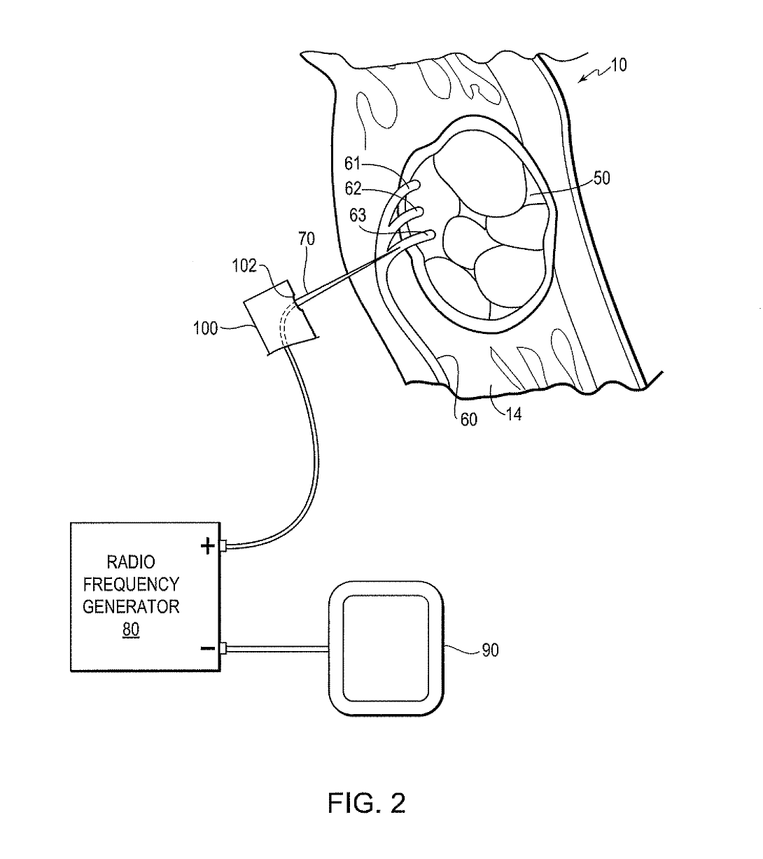

As shown in FIG. 2, a cannula device, for example, an endoscope 100 is used to introduce the needle probe 70 to the location of the fibroid. The endoscope includes a first passage through which the needle probe is inserted. The first passage includes a distal opening 102 at or near the distal end of the endoscope 100, and the distal end of the needle probe 70 can be manipulated by the surgeon to extend from the distal opening 102 of the first passage. The endoscope also typically will include additional passages through which other devices can be introduced to the surgical site. For example, forceps used to temporarily occlude blood vessels can be introduced through another passage of the endoscope 100. In addition, an optical system and/or an imaging system (such as an ultrasonic transducer) can be provided near the distal end of the endoscope, or provided as separate devices that are introduced to the surgical site through passages of the endoscope. The optical system and/or imaging system is/are used by the surgeon during the procedure to monitor the position of, and thereby precisely position, the needle probe as described below.

In addition, an external imaging system, for example, an ultrasonic transducer placed externally on the patient's body, can be used by the surgeon to monitor the position of the needle probe during the procedure.

As an alternative to using an endoscope to introduce the needle probe 70, the probe 70 could be delivered transcutaneously or through its own cannula. It also is possible to use the probe 70 in a transcutaneous procedure in which visualization is all that is needed (if the surgeon is highly skilled and experienced). Fluoroscopy also could be used.

FIG. 2 illustrates the inventive procedure being performed on an intra-mural fibroid 50 located within a wall 14 of the uterus 10. The intra-mural fibroid 50 is supplied with blood by blood vessels 61, 62 and 63. Furthermore, feeder vessel 60 supplies blood to vessels 61-63.

FIG. 2 also shows needle probe 70 extending distally from opening 102 provided near the distal end of an endoscope 100. The needle probe 70 typically has a diameter of 1-2 mm and a pointed distal tip. The pointed distal tip is sufficiently sharp to pierce through wall 14 of uterus 10 and pierce through the blood vessels 60-63. As shown in FIG. 2, by piercing through feeder vessel 60, the distal end of needle probe 70 can be positioned within blood vessel 63. If the needle probe 70 is electrically conductive, the distal end of the needle probe 70 can function as an electrode. In such a case, the outer surface of the needle probe 70 except for the distal end (or a section near the distal end) is covered with an insulative layer so that energy provided through the needle probe 70 is emitted only at the exposed portion of the electrically-conductive needle probe 70. Alternatively, a distinct electrode can be formed on the outer surface of the needle probe, for example, if the material of the needle probe 70 is not electrically conductive, by coating a portion of the surface of the distal end of the needle probe with an electrically conductive material. The electrode portion of the needle probe 70 is attached to the positive output of a radio frequency generator 80. A return electrode 90, which can be a patch electrode, is electrically connected to the negative output (also called the “dispersive pad output” as the signal is an AC signal) of the radio frequency generator 80, and is attached to an external surface of the patient's body, for example, in the abdominal area near the site of the surgery.

Once the distal tip of the needle probe 70 is positioned within the blood vessel (blood vessel 63 in FIG. 2), the radio frequency generator 80 is activated to supply ablative energy to the electrode of the needle probe 70. The supplied ablative energy is sufficient to ablate vessel 63, but is not strong enough (is not applied for a time period long enough) to damage surrounding tissue of the uterine wall 14 or the fibroid 50. The supplied energy can be pulsed to prevent excessive heat build-up. The amount of power supplied varies based on numerous factors such as, for example, vessel size, surrounding blood flow rates, the use of pulsing, the desired duration of the procedure, etc. Typically from about 2 W to about 35 W is supplied. Because the needle probe includes only an active electrode (that is, the return electrode 90 is provided separate from the probe 70), the electrode on the needle probe 70 can be referred to as a “monopolar electrode.”

Once the surgeon has confirmed that blood vessel 63 has been ablated, a similar procedure can be performed on the remaining blood vessels, such as vessels 61 and 62 in the FIG. 2 example. Alternatively, if possible, feeder vessel 60 could be ablated instead of ablating each of its fed vessels 61-63. In general, the vessels specifically feeding a fibroid are smaller than about 4 mm in diameter.

According to some embodiments, the supply of blood to the vessel(s) to be ablated can be blocked prior to performing the ablation procedure in order to minimize the loss of blood that can occur when the needle probe 70 pierces the blood vessel. For example, according to one embodiment, the blood vessel (for example, vessel 60 in FIG. 2) could be occluded by applying external pressure to the blood vessel 60. For example, a forceps or other force-applying device could be located on the surface of wall 14 near vessel 60, and then used to apply force to the vessel 60 (through the wall 14) so as to partially or completely occlude the vessel 60.

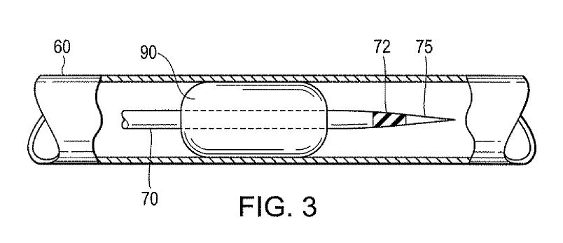

Another alternative to occluding the blood vessel would be to deploy an occlusive device from the needle probe 70 after the needle probe has pierced the blood vessel. FIG. 3 illustrates an example of a needle probe 70 that can be used to also occlude the vessel into which it has been inserted. The needle probe 70 includes sharp distal tip 75 and a monopolar electrode 72 provided on the distal tip 75. A conductive wire would attach the monopolar electrode 72 to the radio frequency generator 80. In addition, an inflatable balloon 90 is provided on the external surface of the needle probe 70. FIG. 3 shows balloon 90 in the inflated state. The balloon 90 would be deflated until after the needle probe 70 has pierced the artery 60 and been placed at the desired location within the artery 60. Then, gas is supplied to the inside the balloon 90 through a passage (not shown) through the needle probe 70 so as to inflate the balloon 90 and thereby occlude the vessel 60. Subsequent to occlusion, ablative energy is provided to the monopolar electrode 72 (either continuously or pulsed) until the artery 60 has been occluded. The balloon then is deflated, and the needle probe 70 is retracted from the surgical site and into the endoscope 100.

Once the blood vessels feeding a fibroid have been ablated, the electrode of the needle probe 70 can be activated as it is withdrawn from the puncture site to prevent bleeding (this is referred to as “track ablation”). A suture could be used to close the puncture site if necessary or desired. If necessary, a stitch could be used to close the uterine wall, but it is unlikely that the use of a stitch would be necessary assuming no punctures have been made entirely through the uterine wall.

None of the embodiments require excision of the uterine fibroid, which will eventually necrose and shrink without further surgical intervention.

The illustrated exemplary embodiments are intended to be illustrative and not limiting. Various changes may be made without departing from the spirit and scope of the invention. For example, the method may be performed on any type of uterine fibroid.

What is claimed is:

1. A method of treating a uterine fibroid, the method comprising:
(i) piercing a blood vessel that supplies blood to the fibroid with a sharp distal end of a needle probe, the needle probe including an electrically conductive electrode located at and/or adjacent to the distal end of the needle probe;
(ii) positioning the electrode within the blood vessel; and
(iii) supplying energy to the electrode to ablate the blood vessel and thereby destroy the supply of blood to the fibroid through the blood vessel.

2. The method according to claim 1, further comprising performing steps (i)-(iii) for all blood vessels that supply blood to the fibroid.

3. The method according to claim 1, wherein the energy is monopolar energy and a return electrode is attached to the patient having the fibroid at a position on the patient located distant from the distal end of the needle probe.

4. The method according to claim 1, wherein the distal end of the needle probe has a diameter of about 1-2 mm.

5. The method according to claim 1, further comprising temporarily blocking flow of blood through the blood vessel before the step of piercing the blood vessel.

6. The method according to claim 5, wherein the step of temporarily blocking the flow of blood through the blood vessel includes compressing the blood vessel.

7. The method according to claim 1, further comprising temporarily blocking flow of blood through the blood vessel after step (i) but before step (iii) by deploying a balloon in the blood vessel.

8. The method according to claim 7, wherein the balloon is deployed from the needle probe.

9. The method according to claim 1, wherein an imaging technique is utilized to guide the distal end of the needle probe to the blood vessel.

10. The method according to claim 1, wherein step (iii) ablates the blood vessel without causing ablation to occur in the fibroid.
